(19) United States
(12) Patent Application Publication
(10) Pub. No.: US 2015/0031097 A1
(43) Pub. Date: Jan. 29, 2015

(54) MOBILE PROCESSING SYSTEMS AND METHODS FOR PRODUCING BIODIESEL FUEL FROM WASTE OILS

(71) Applicant: Revolution Fuels, Inc., Minneapolis, MN (US)

(72) Inventors: James M. Rethore, Forest, VA (US); Julie E. Wheeler, Minneapolis, MN (US); Luca Zullo, Excelsior, MN (US)

(21) Appl. No.: 14/374,224

(22) PCT Filed: Jan. 30, 2013
(86) PCT No.: PCT/US2013/023852

§ 371 Date: Jul. 23, 2014

Related U.S. Application Data
(60) Provisional application No. 61/594,301, filed on Feb. 2, 2012.

(51) Int. Cl. C12P 7/64; H01M 8/06; C11C 1/08; F02B 63/04; C11C 3/00; C11C 3/10

(52) U.S. Cl. CPC C12P 7/649; C11C 3/003; C11C 3/10; C11C 1/08; F02B 63/04; H01M 8/0612
USPC 435/134; 554/165; 60/780; 429/416

(57) ABSTRACT

The present invention improves biodiesel production in several ways. Unique combinations of unit operations and flow configurations are disclosed in mobile processing units that are feedstock-flexible and can be dynamically deployed in a distributed way. In some embodiments, a process includes introducing a waste oil and an alcohol into a reactor with an esterification-transesterification enzymatic catalyst. Free fatty acids are reacted with alcohol to produce fatty acid alkyl esters, and glycerides are reacted with alcohol to produce fatty acid alkyl esters and glycerin. A membrane separator removes glycerin, water, and alcohol. Unreacted free fatty acids are then separated and recycled, to generate a product stream with fatty acid alkyl esters. A genset may be provided for combusting glycerin to produce electrical power and thermal heat as co-products. This biodiesel process may be energy self-sufficient, require no external utilities, and avoid direct discharge of wastewater.

### PRIORITY DATA

This international patent application claims priority to U.S. Patent App. No. 61/594,301, filed Feb. 2, 2012 for MOBILE PROCESSING SYSTEMS AND METHODS FOR PRODUCING BIODIESEL FUEL FROM WASTE OILS, which is hereby incorporated by reference herein.

### FIELD OF THE INVENTION

The present invention generally relates to methods and systems for the conversion of waste oils, such as used cooking oil, into biodiesel fuel.

### BACKGROUND OF THE INVENTION

Biodiesel fuel is a diesel fuel consisting of long-chain alkyl esters, derived from vegetable oil or animal fat. Biodiesel fuel can be made by chemically reacting lipids with an alcohol, typically methanol or ethanol. Biodiesel fuel can be used in standard diesel engines, either alone or blended with petroleum-derived diesel fuel. Biodiesel fuel can also be used as a low-carbon alternative to heating oil.

Biodiesel fuel is a renewable fuel with lower emissions than those associated with petroleum-derived diesel fuel. Since the passage of the Energy Policy Act of 2005, biodiesel fuel use has been increasing in the United States. Biodiesel fuel has virtually no sulfur content, and it is often used as an additive to Ultra-Low Sulfur Diesel fuel. Biodiesel fuel has much higher cetane ratings than lower-sulfur diesel fuels. Biodiesel fuel addition reduces fuel system wear, and can increase the life of fuel-injection equipment that relies on the fuel for its lubrication.

There is significant global interest in producing fuels and chemicals from renewable resources. Concurrently, there is a driver to utilize non-food feedstocks and other waste materials that do not cause supply concerns within food business systems. In the context of biodiesel fuels, there is an interest in using waste grease, rather than fresh vegetable oil, to make biodiesel fuel. This approach can essentially double the productive value of vegetable oil, and not use a food (fresh oil) as a fuel.

The cooking-oil industry, and the food industry in general, desires clean sources of energy; lower-cost alternatives to petroleum-derived diesel fuel; the ability to convert waste into value; and improved environmental life cycles in the supply chain, including lower fuel use and lower total carbon emissions. Improved processes and systems are needed for converting waste oil, and other oils, fats, and greases, into biodiesel fuel.

One possible way of improving the process is to employ enzymatic esterification and transesterification catalysts. Enzymatic biodiesel production has been investigated, but is presently not employed industrially in any significant way. The advantages of enzymes remain merely theoretical. See Fjerbaek et al., “A Review of the Current State of Biodiesel Production Using Enzymatic Transesterification,” Biotechnology and Bioengineering, Vol. 102, No. 5, Apr. 1, 2009, which is hereby incorporated by reference herein.

There remains a need to improve biodiesel production, including by selecting unit operations and flow configurations that enable the efficient use of enzymes. It would be especially attractive for a process to be mobile and feedstock-flexible, so that it can be effectively deployed in a distributed and dynamic way.

### SUMMARY OF THE INVENTION

In some variations, this invention provides a process for producing biodiesel fuel, the process comprising:

(a) providing a feed stream comprising glycerides and free fatty acids;

(b) optionally filtering out at least a portion of solids contained in the feed stream;

(c) introducing the feed stream and an alcohol into a first reactor, operated at effective first reaction conditions and including a first effective esterification and transesterification catalyst, or mixture of such catalysts, to react at least a portion of the free fatty acids with the alcohol to produce fatty acid alkyl esters and water, and to react at least a portion of the glycerides with the alcohol to produce fatty acid alkyl esters and glycerin, thereby producing a first intermediate stream comprising fatty acid alkyl esters, glycerin, water, and alcohol;

(d) introducing at least a portion of the first intermediate stream to a first separator operated to remove at least some glycerin, water, and alcohol, thereby producing a second intermediate stream comprising fatty acid alkyl esters and unreacted free fatty acids;

(e) optionally further reacting, separating, or treating the second intermediate stream; and

(f) introducing the second intermediate stream and a base to a fatty acid alkyl ester recovery unit operated to contact the base with the unreacted free fatty acids to produce a fatty acid-base complex, and separate the fatty acid-base complex from the fatty acid alkyl esters, thereby generating a crude product stream comprising the fatty acid alkyl esters.

Generally, glycerides include monoglycerides, diglycerides, and triglycerides. In some embodiments, the glycerides consist essentially of triglycerides.

The feed stream may include an oil derived from a food source selected from the group consisting of soybeans, corn, canola, rice, olive, coconut, cottonseed, palm, peanut, rapeseed, safflower, sesame, sunflower, pumpkin, grape, animal fat, and combinations thereof.

In some embodiments, the feed stream includes a waste oil. In some embodiments, the feed stream includes an inedible oil. Waste oils or inedible oils may be derived from energy crops, in certain embodiments.

The alcohol may be selected from the group of C₁-C₆ alcohols, and combinations thereof. In certain embodiments, the alcohol is methanol.

The process may comprise intensively mixing the alcohol with the feed stream and with recycled free fatty acids, if any. Intensive mixing may be carried out using an in-line mixer, a mixing tank, or a colloid mill, for example.

In some embodiments, the first effective esterification and transesterification catalyst is a single catalyst with both esterification and transesterification activity. Or, the first effective esterification and transesterification catalyst may be a mixture of catalysts collectively including esterification and transesterification activity. In some embodiments, the first effective esterification and transesterification catalyst is an alkali catalyst or an acid catalyst.

In preferred embodiments, the first effective esterification and transesterification catalyst is an enzymatic catalyst with activity, at the effective first reaction conditions, selected from the group consisting of lipase activity, phospholipase activity, esterase activity, and combinations thereof. In some embodiments, the enzymatic catalyst is immobilized. Preferably, the enzymatic catalyst is tolerant to the alcohol employed.

The first reactor may be a continuous stirred-tank reactor or a fixed-bed reactor, for example. The first separator may be selected from the group consisting of a distillation unit, a flash evaporation unit, a centrifuge, a filter, membranes, and any combinations thereof. In preferred embodiments, the first separator comprises membranes.

The fatty acid alkyl ester recovery unit may be selected from the group consisting of a distillation unit, a flash evaporation unit, a centrifuge, a filter, membranes, and any combinations thereof. In preferred embodiments, the fatty acid alkyl ester recovery unit utilizes several membranes in an integrated fashion. For example, the fatty acid alkyl ester recovery unit may includes a first membrane contactor, a membrane separator for separating the fatty acid alkyl esters from the fatty acid-base complex, and a second membrane contactor for recovering the base. The base may consist essentially of ammonia and/or derivatives thereof (e.g., ammonium hydroxide). The base may then be recovered from the fatty acid-base complex to generate free fatty acids.

In some embodiments, the process further comprising introducing glycerin, water, and alcohol from the first separator to an alcohol recovery unit operated to remove alcohol, thereby producing a glycerin stream comprising glycerin and water. The alcohol recovery unit may be selected from the group consisting of a distillation unit, a flash evaporation unit, a centrifuge, membranes, molecular sieves, and any combinations thereof. Some or all of the alcohol removed from the alcohol recovery unit may be recycled back to step (c), either directly into the first reactor or to the location for intensive mixing with the feed stream.

In some embodiments, the process further comprises combusting at least some of the glycerin stream in a genset to produce electrical power and thermal heat. In certain embodiments, both glycerin and alcohol from the first separator may be introduced to a genset to produce electrical power and thermal heat. In these or other embodiments, glycerin, alcohol, and water from the first separator may be introduced to a fuel cell genset to produce electrical power and thermal heat.

The process further comprises conveying the crude product stream to storage, to a point of use, or to further upgrading or polishing. For example, the crude product stream may be sent to one or more polishing steps to produce a product stream comprising the fatty acid alkyl esters. In some embodiments, the polishing steps include ion exchange.

Other variations of the invention provide a process for producing biodiesel fuel, the process comprising:

(a) providing a feed stream comprising glycerides and free fatty acids;

(b) optionally filtering out at least a portion of solids contained in the feed stream;

(c) introducing the feed stream and an alcohol into a first reactor, operated at effective first reaction conditions and including a first effective esterification and transesterification catalyst, or mixture of such catalysts, to react at least a portion of the free fatty acids with the alcohol to produce fatty acid alkyl esters and water, and to react at least a portion of the glycerides with the alcohol to produce fatty acid alkyl esters and glycerin, thereby producing a first intermediate stream comprising fatty acid alkyl esters, glycerin, water, and alcohol;

(d) introducing at least a portion of the first intermediate stream to a first separator operated to remove at least some glycerin, water, and alcohol, thereby producing a second intermediate stream comprising fatty acid alkyl esters and unreacted free fatty acids;

(e) introducing the second intermediate stream and an alcohol to a second reactor, operated at effective second reaction conditions and including a second effective esterification and transesterification catalyst, or mixture of such catalysts, to react free fatty acids contained therein with the alcohol to produce fatty acid alkyl esters and water, and/or to react glycerides contained therein with the alcohol to produce fatty acid alkyl esters and glycerin, thereby producing a third intermediate stream comprising fatty acid alkyl esters, glycerin, water, and alcohol;

(f) introducing at least a portion of the third intermediate stream to a second separator operated to remove at least some glycerin, water, and alcohol, thereby producing a fourth intermediate stream comprising fatty acid alkyl esters and unreacted free fatty acids; and

(g) introducing the fourth intermediate stream and a base to a fatty acid alkyl ester recovery unit operated to contact the base with the unreacted free fatty acids to produce a fatty acid-base complex, and separate the fatty acid-base complex from the fatty acid alkyl esters, thereby generating a crude product stream comprising the fatty acid alkyl esters.

The second effective esterification and transesterification catalyst may be a single catalyst with both esterification and transesterification activity, or a mixture of catalysts collectively including esterification and transesterification activity. The first and second effective esterification and transesterification catalysts may be the same.

In some embodiments, the second effective esterification and transesterification catalyst is an alkali catalyst. In other embodiments, the second effective esterification and transesterification catalyst is an acid catalyst.

In preferred embodiments, the second effective esterification and transesterification catalyst is an enzymatic catalyst with activity, at the effective first reaction conditions, selected from the group consisting of lipase activity, phospholipase activity, esterase activity, and combinations thereof. The second effective esterification and transesterification catalyst may be immobilized, and may be selected for its tolerance to the alcohol, in some embodiments.

Similar to the first reactor, the second reactor may be a continuous stirred-tank reactor or a fixed-bed reactor, for example. In some embodiments, the second separator is selected from the group consisting of a distillation unit, a flash evaporation unit, a centrifuge, a filter, membranes, and any combinations thereof. Preferably, the second separator comprises membranes.

Certain variations of the invention provide a process for producing biodiesel fuel from free fatty acids, the process comprising:

(a) providing a feed stream comprising glyceride-derived free fatty acids, without triglycerides substantially present;

(b) introducing the free fatty acids and an alcohol into a first reactor, operated at effective first reaction conditions and including a first effective esterification catalyst, to react at least a portion of the free fatty acids with the alcohol to produce fatty acid alkyl esters and water, thereby producing a first intermediate stream comprising fatty acid alkyl esters, water, and alcohol;

(c) introducing at least a portion of the first intermediate stream to a first separator operated to remove at least some water and alcohol, thereby producing a second intermediate stream comprising fatty acid alkyl esters and unreacted free fatty acids;

(d) optionally introducing the second intermediate stream and an alcohol to a second reactor, operated at effective second reaction conditions and including a second effective esterification catalyst, to react free fatty acids contained therein with the alcohol to produce fatty acid alkyl esters and water, thereby producing a third intermediate stream comprising fatty acid alkyl esters, water, and alcohol;

(e) optionally introducing at least a portion of the third intermediate stream, if produced by step (d), to a second separator operated to remove at least some water and alcohol, thereby producing a fourth intermediate stream comprising fatty acid alkyl esters and unreacted free fatty acids; and

(f) introducing the second intermediate stream and/or the fourth intermediate stream, if the fourth stream is produced by steps (d)-(e), and a base to a fatty acid alkyl ester recovery unit operated to contact the base with the unreacted free fatty acids to produce a fatty acid-base complex, and separate the fatty acid-base complex from the fatty acid alkyl esters, thereby generating a crude product stream comprising the fatty acid alkyl esters.

The alcohol may be selected from the group of C₁-C₆ alcohols, and combinations thereof. In some embodiments, the alcohol is methanol. The process may include intensively mixing the alcohol with the free fatty acids, prior to or during step (b).

In some embodiments, the first effective esterification catalyst is a single catalyst with esterification activity, or a mixture of catalysts collectively including esterification activity. It is noted that in this variation, the catalyst does not need to have transesterification activity. The first effective esterification catalyst may be an alkali or acid catalyst. In preferred embodiments, the first effective esterification catalyst is an enzymatic catalyst with activity, at the effective first reaction conditions, selected from the group consisting of lipase activity, phospholipase activity, esterase activity, and combinations thereof. Again, the enzyme may be immobilized, and it may be tolerant to alcohol (e.g., methanol).

When steps (d) and (e) are carried out, the first effective esterification catalyst may be the same as the second effective esterification catalyst. In other embodiments, the first and second esterification catalysts are not the same.

The fatty acid alkyl ester recovery unit may be selected from the group consisting of a distillation unit, a flash evaporation unit, a centrifuge, a filter, membranes, and any combinations thereof. In preferred embodiments, the fatty acid alkyl ester recovery unit utilizes several membranes in an integrated fashion. For example, the fatty acid alkyl ester recovery unit may includes a first membrane contactor, a membrane separator for separating the fatty acid alkyl esters from the fatty acid-base complex, and a second membrane contactor for recovering the base. The base may consist essentially of ammonia and/or derivatives thereof (e.g., ammonium hydroxide). The base may then be recovered from the fatty acid-base complex to generate free fatty acids. These free fatty acids may be recycled and combined with the fresh free fatty acids in the feed stream.

The process of this variation further comprises conveying the crude product stream to storage, to a point of use, or to further upgrading or polishing. For example, the crude product stream may be sent to one or more polishing steps to produce a product stream comprising the fatty acid alkyl esters. In some embodiments, the polishing steps include ion exchange.

Still other variations of the invention are premised on the combination of reaction and separation in membrane reactors. In some embodiments, a process for producing biodiesel fuel comprises:

(a) providing a feed stream comprising glycerides and free fatty acids;

(b) optionally filtering out at least a portion of solids contained in the feed stream;

(c) introducing the feed stream and an alcohol into a membrane reactor configured with immobilized esterification and transesterification enzymes supported on membranes, wherein the membrane reactor is operated at effective reaction conditions to react at least a portion of the free fatty acids with the alcohol to produce fatty acid alkyl esters and water, and to react at least a portion of the glycerides with the alcohol to produce fatty acid alkyl esters and glycerin, thereby producing an in situ mixture comprising fatty acid alkyl esters, glycerin, water, and alcohol; and wherein the membrane reactor, under effective reaction conditions, continuously separates at least some glycerin, water, and alcohol, thereby producing an intermediate stream comprising fatty acid alkyl esters and unreacted free fatty acids;

(d) optionally further reacting, separating, or treating the intermediate stream; and

(e) introducing the intermediate stream and a base to a fatty acid alkyl ester recovery unit operated to contact the base with the unreacted free fatty acids to produce a fatty acid-base complex, and separate the fatty acid-base complex from the fatty acid alkyl esters, thereby generating a crude product stream comprising the fatty acid alkyl esters.

In some embodiments of the invention, a process as disclosed herein is disposed substantially on a mobile processing unit. The process may continuous, semi-continuous, or a batch process. The process may be designed to converts between 100 and 10,000 gallons per hour of the feed stream, such as 1,000 gallons per hour of the feed stream, or more. In some embodiments, substantially no wastewater is directly discharged in connection with production of the biodiesel fuel. The process is energy self-sufficient and requires substantially no external utilities, in specific embodiments.

Some variations of the invention provide systems configured for carrying out any of the described processes. Other variations of this invention provide biodiesel fuel compositions produced by any of the described processes.

### BRIEF DESCRIPTION OF THE FIGURES

FIG. 1 is a simplified block-flow diagram depicting some variations of the invention for converting oils, fats, and grease into biodiesel fuel.

FIG. 2 is a simplified block-flow diagram depicting some variations of the invention for converting oils, fats, and grease into biodiesel fuel, as well as power production from the glycerin co-product.

FIG. 3 is a block-flow diagram depicting certain embodiments of the invention for converting waste vegetable oil into biodiesel fuel and power.

FIG. 4 is a block-flow diagram depicting certain embodiments of the invention for converting waste vegetable oil into biodiesel fuel.

### DETAILED DESCRIPTION OF EMBODIMENTS OF THE INVENTION

Certain embodiments of the present invention will now be further described in more detail, in a manner that enables the claimed invention so that a person of ordinary skill in this art can make and use the present invention. These and other embodiments, features, and advantages of the present invention will become more apparent to those skilled in the art when taken with reference to the following detailed description of the invention in conjunction with the accompanying drawings.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in its respective testing measurements.

As used in this specification and the appended claims, the singular forms “a,” “an,” and “the” include plural referents unless the context clearly indicates otherwise. Any reference to “comprising” includes, in the alternative, “consisting essentially of” or “consisting of” in certain embodiments. Unless defined otherwise, all technical and scientific terms used herein have the same meaning as is commonly understood by one of ordinary skill in the art to which this invention belongs. If a definition set forth in this section is contrary to or otherwise inconsistent with a definition set forth in patents, published patent applications, and other publications that are herein incorporated by reference, the definition set forth in this specification prevails over the definition that is incorporated herein by reference.

As used herein, “FFA” is free fatty acid; “TAG” is triacylglyceride (or equivalently, triglyceride); “FAAE” is fatty acid alkyl ester; and “FAME” is fatty acid methyl ester.

“Biodiesel fuel” as intended herein is meant to be broadly construed to include liquid fuels for vehicles that travel over land (e.g., trucks), rail (e.g., trains), water (e.g., ships), and air (e.g., aircraft). Biodiesel fuel also can be suitable for stationary power applications, such as fuel oil or gas turbine fuel. For any of these applications, biodiesel fuel can be used alone or in blended form with petroleum-derived diesel fuel, or with another source of diesel fuel such as Fischer-Tropsch fuels from synthesis gas.

Some variations of the present invention are premised on the realization that certain novel and non-obvious process-flow configurations are particularly effective for the conversion of oil (including fats, grease, waste oil, etc.) into biodiesel fuel.

Embodiments, features, and advantages of the present invention will become apparent to those skilled in the art when taken with reference to the following detailed description of the invention in conjunction with the accompanying drawings. The drawings are exemplary of some embodiments only and do not limit the scope of the claimed invention.

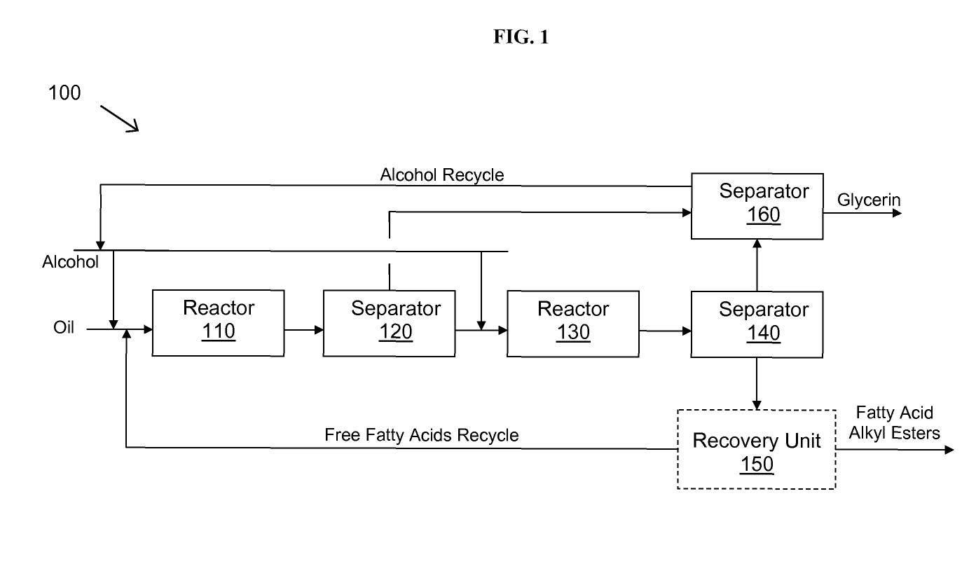

With reference to FIG. 1, a process 100 is provided wherein an oil feed stream that includes glycerides and free fatty acids is converted into fatty acid alkyl esters, which are suitable as components for biodiesel fuels.

“Glycerides,” also known as acylglycerols or acylglycerides, are esters formed from glycerin (also known as glycerol) and fatty acids. Glycerin has three hydroxyl functional groups, which can be esterified with one, two, or three fatty acids to form monoglycerides, diglycerides, and triglycerides. Fatty acids are aliphatic compounds containing 4 to 24 carbon atoms and having a terminal carboxyl group. Diglycerides are esters of glycerol and two fatty acids, and monoglycerides are esters of glycerol and one fatty acid. Naturally occurring fatty acids, with minor exceptions, have an even number of carbon atoms. Triglycerides are found in a large variety of fats and oils, including natural oils as well as industrial and commercial waste oils (e.g., restaurant grease). Partial glycerides are esters of glycerol with fatty acids, where not all the hydroxyl groups are esterified. For the purposes of this disclosure, glycerides include partial glycerides.

In some embodiments of the invention, the oil is waste oil. “Waste oil” should be broadly construed herein to include waste vegetable oil, animal fats, various edible or inedible oils, or combinations of fresh and used oil. The principles of the present invention may be applied to any oil source, i.e. any fat, oil, or grease stream or combinations thereof. Of course, the invention may be utilized with fresh or virgin oil obtained from a vegetable or animal source. Typically, the oil utilized will contain at least some free fatty acids. Lower-quality oil streams tend to have higher free fatty acid content. The invention can deal with high concentrations of free fatty acids in the feed; in some embodiments, the oil may consist entirely of fatty acids.

For example, the oil may be derived from a food source selected from soybeans, corn, canola, rice, olive, coconut, cottonseed, palm, peanut, rapeseed, safflower, sesame, sunflower, pumpkin, grape, animal fat, or combinations thereof. Any type of animal fat may be used, including rendered fat, tallow, lard, etc.

The oil may be derived from an energy crop, if desired. For example, the oil may be derived from one or more of copra, castor seed, sesame, groundnut kernel, jatropha, rapeseed, palm kernel, mustard seed, sunflower, palm fruit, soybean, or cotton seed. An energy crop is a low-cost plant grown to produce at least energy in some form. Energy crops are not necessarily precluded from uses in food, and generally speaking, edible cooking oil may be extracted from an energy crop. For example, an energy crop may provide fermentable sugars for producing ethanol, while at the same time, provide extractable oil which may be converted into biodiesel fuel, either directly or following use as cooking oil.

The oil feed stream may be first heated, filtered, and/or otherwise treated prior to introduction to reactor 110 (FIG. 1). For example, the oil may be heated to improve flowability by passing the oil through a heat exchanger (e.g., a plate and frame heat exchanger). The oil or heated oil may also be filtered to remove any foreign material. For example, the oil may be passed through an in-line liquid filter bag (e.g., a 1-micron filter bag), filter press, or other type of filter.

The alcohol may be selected from C₁-C₆ alcohols, and any combinations thereof, including any isomers of these alcohols. In some embodiments, the alcohol is methanol. In some embodiments, the alcohol is a mixture of methanol and ethanol. In some embodiments, the alcohol includes 1-butanol and/or isobutanol. In certain embodiments, a mixed-alcohols stream is utilized, such as obtained from mixed-alcohol synthesis from syngas. Such a mixed-alcohols source will typically include primarily methanol and ethanol, but also higher alcohols including propanol, butanol, pentanol, and hexanol, and possibly small amounts of larger alcohols.

When the alcohol reactant is obtained from a renewable resource (e.g., cellulosic ethanol or butanol from sugar fermentation), an additional amount of Renewable Identification Numbers (RINs) or tax credits may be generated in connection with the biodiesel fuel produced. Thus, C₂ or higher alcohol reactants may be desired since methanol cannot be obtained directly from sugar fermentation.

The feed oil along with any recycled oil is mixed with the alcohol, preferably at or near stoichiometric levels for the esterification and transesterification chemistry. In preferred embodiments, the oil is intensively mixed with the alcohol prior to introduction in reactor 110.

By “intensively mixed” or “intensive mixing” or the like, it is meant that the components are blended in a manner that achieves an intimate association of alcohol and glycerides and/or fatty acids. Such intensive mixing may be accomplished using various means, such as an in-line mixer, a mixing tank, or colloid mill, for example. A colloid mill applies high levels of hydraulic shear to the process liquid.

Reactor 110 is operated under effective conditions suitable for esterification and/or transesterification to take place. Esterification converts an acid (e.g., a free fatty acid) to an ester (e.g., a fatty acid alkyl ester) by reaction with an alcohol, generating water in the process. Transesterification converts one ester into another ester by reaction with an alcohol. When transesterification converts triglycerides (in the oil) to fatty acid alkyl esters, glycerin is also produced.

Reactor 110 may be any type of reactor suitable for carrying out esterification and transesterification. Preferably, the reactor is a closed reaction vessel, to prevent loss of alcohol to the atmosphere. The reactor can be engineered and operated in a wide variety of ways. The temperature and residence time of reactor 110 will be dictated by the choice of catalyst(s) for this reactor, as discussed below. The reactor operation can be continuous, semi-continuous, pseudo-continuous, batch, or some combination or variation of these operating modes, but it is preferably continuous or at least semi-continuous. Operation that is continuous and at steady state is preferable. The flow pattern can be substantially plug flow, substantially well-mixed, or a flow pattern between these extremes. The flow direction can be vertical-upflow, vertical-downflow, or horizontal. Any “reactor” herein can in fact be a series or network of several reactors in various arrangements.

The esterification and transesterification may be catalyzed with one or more acids or bases. It is generally preferred to employ bases over acids, as is known in the art, due to lower temperatures (and therefore pressures) possible, higher yields, reduced side reactions, and less-expensive materials of construction. Base catalysts can be strong alkali catalysts, such as sodium methoxide (also known as sodium methylate, CH₃ONa), sodium hydroxide, or potassium hydroxide. Acid catalysts can be, for example, sulfuric acid, hydrochloric acid, and other acids.

Thus in some embodiments, reactor 110 is a continuous stirred-tank reactor (CSTR) with an acid or base catalyst that is present in solution. The residence time of the CSTR may vary widely, such as from about 0.1 hr to about 24 hr, e.g. from about 1 hr to about 4 hr.

In some embodiments, reactor 110 is a continuous stirred-tank reactor (CSTR) with enzymes that are present in solution, preferably on support particles that are restricted from exiting the CSTR (e.g., using a screen). The residence time of the CSTR may vary widely, such as from about 0.1 hr to about 24 hr, e.g. from about 1 hr to about 4 hr. The reaction temperature may vary between about 70-160° F., for example.

Alternatively, reactor 110 may be a packed-bed reactor or a trickle-bed reactor. A packed-bed reactor may include immobilized catalyst (e.g., enzymes) on support structures (e.g., packing beads) or the walls of the reactor, for example.

The output from reactor 110 is directed to separator 120, wherein glycerin, water, and alcohol are at least partially separated from the FAME/FFA/TAG stream. Separator 120 may be selected from any known separation device, such as a distillation unit, a flash evaporation unit, a centrifuge, a filter, membranes, or any combinations thereof. Separation can be principally based, for example, on distillation, absorption, adsorption, or diffusion, and can utilize differences in vapor pressure, activity, molecular weight, density, viscosity, chemical functionality, and any combinations thereof. In preferred embodiments, separator 120 is a membrane separator (further discussion of membranes is included below).

In some embodiments, the FAME/FFA/TAG stream is then mixed with additional alcohol and directed to a second reactor 130. A second reactor is useful because typically the first reactor is limited by the equilibrium conversion of free fatty acids to alkyl esters (e.g., about 90% conversion). Water formation prevents further conversion. A second reactor is not strictly necessary, however, if the conversion attained in a single reactor is acceptable, or if water is continuously removed. In certain embodiments that employ enzyme-coated membranes, at least a portion of reaction products (e.g., water or glycerin) are continuously removed. In this case the equilibrium conversion does not constrain the reaction, and essentially complete conversion can in principle be achieved.

Reactor 130 is operated under effective conditions suitable for esterification and/or transesterification to take place. All of the reaction-engineering considerations described above for reactor 110 are applicable to reactor 130 as well. Generally speaking, although reactor 130 may be similar to reactor 110, that is not at all necessary. All parameters for the design and operation of reactor 130 may be independently selected versus reactor 110, when possible.

The output from reactor 130 (when present) is directed to a second separator 140, wherein once again glycerin, water, and alcohol are at least partially separated from the finished FAME/FFA stream exiting reactor 130. If the second reactor 130 is not present or is being bypassed, then the second separator 140 is optional (although it still may be employed).

The fatty acid alkyl esters, which are biodiesel components, need to be separated from the unreacted free fatty acids (or other fatty acids present). The separation of free fatty acids from fatty acid alkyl esters is accomplished in fatty acid alkyl ester recovery unit 150, which may include a distillation unit, a flash evaporation unit, a centrifuge, or plurality of membranes, for example. Preferably, membranes are employed.

The fatty acid alkyl ester recovery unit 150 is preferably operated as a reactive separation unit in which a base (e.g., ammonia) is contacted with unreacted free fatty acids to produce a fatty acid-base complex. A “fatty acid-base complex” means a molecular association where the fatty acid and the base chemically or physically bond, absorb, adsorb, or otherwise interact with each other at the molecular level. The fatty acid-base complex may then be conveniently separated from fatty acid alkyl esters. Then, through heating or other means, the base may be recovered from the fatty acid-base complex to generate free fatty acids for optional recycling.

The fatty acid alkyl ester recovery unit 150 is depicted with dotted lines to indicate that it typically will include several sub-units that are not separately shown in the figure. In certain embodiments, the fatty acid alkyl ester recovery unit 150 includes a first membrane contactor, a membrane separator for separating fatty acid alkyl esters from fatty acid salts, and a second membrane contactor for recovering the base. See the section “Membrane Separators” below for more details on the membranes.

The fatty acid alkyl esters may be directed to finished storage, or directly to a means for transporting or using the product. In some embodiments, the fatty acid alkyl ester stream may be directed through a polishing step comprising, but not limited to, ion exchange.

The glycerin, water, and alcohol streams derived from separator 120 and/or separator 140 are further directed to another separator 160 where the glycerin/water and alcohol are separated. Separator 160 may be selected from, for example, a distillation unit, a flash evaporation unit, a vacuum flash unit, a centrifuge, molecular sieves, or membranes. Various splits and purities of alcohol, water, and glycerin are possible, into two or more streams. The alcohol may be recycled back to the alcohol feed stream, or directly into reactor 110 and/or reactor 130.

The glycerin may be separated and stored, sold, or used for various purposes. For example, the glycerin may be reformed to syngas (which may be used for power), or directly oxidized to generate power. In some embodiments, the glycerin, alone or in combination with unreacted alcohol or with biodiesel fuel, may be reformed or oxidized to generate combined heat and power.

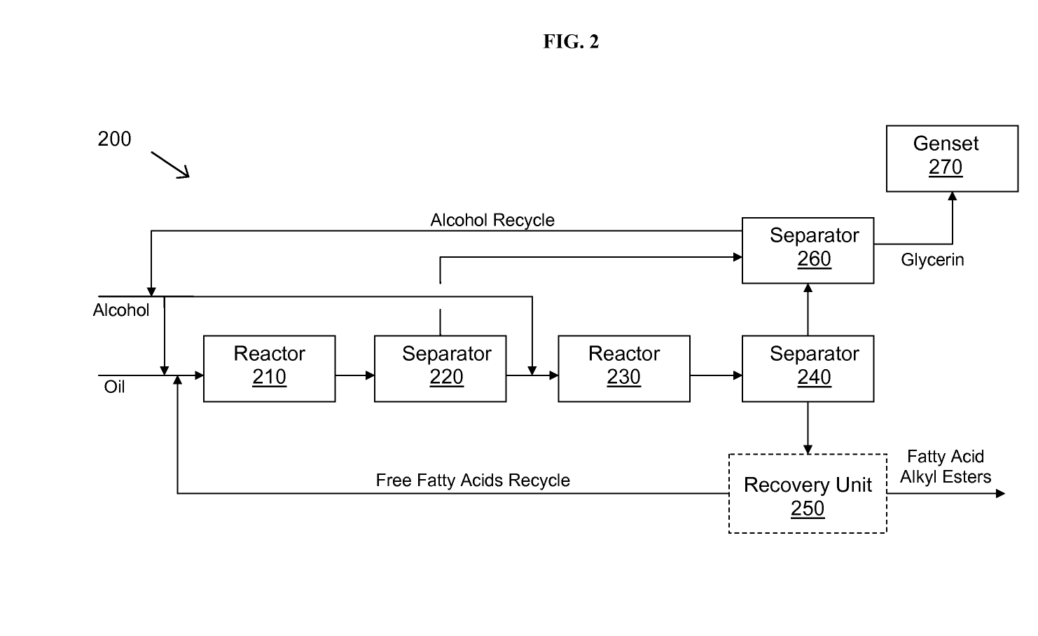

In some embodiments, such as depicted in FIG. 2, the glycerin or glycerin/water is directed to a genset 270 where it may be combusted to produce power. The power produced may be utilized for the process itself and any ancillary power needs, or some power may be exported. The genset 270 may be any known engine-generator, i.e. a combination of an electrical generator and an engine (prime mover) mounted together. The electrical generator may be a fuel cell, to form a fuel cell genset.

In some embodiments, the genset 270 may be powered not only by the glycerin co-produced, but also by a power take-off from an available external system, such as a truck cab. A power take-off would give the genset 270 the ability to be turned mechanically. This may be useful at start-up of the process, since there will not yet be a glycerin flow. The external system may be a cab's transmission's system, for example. In some embodiments, other external power sources (e.g., battery packs) are provided, so that power may be available from the truck until there is sufficient glycerin flow. The truck is preferably the truck that moves the mobile processing unit (see below). In preferred embodiments, the genset 270 is designed and operated so that the overall process requires no local power source.

Any unit can be replicated in parallel, to add capacity to the overall process. For example, any of reactors 110, 210, 130, or 230 can actually be two or more individual reactors. Any of separators 120, 220, 140, or 240 can actually be two or more individual separation units. Engineering and design optimization may be carried out by a person of ordinary skill in the art, to achieve a desired overall process capacity.

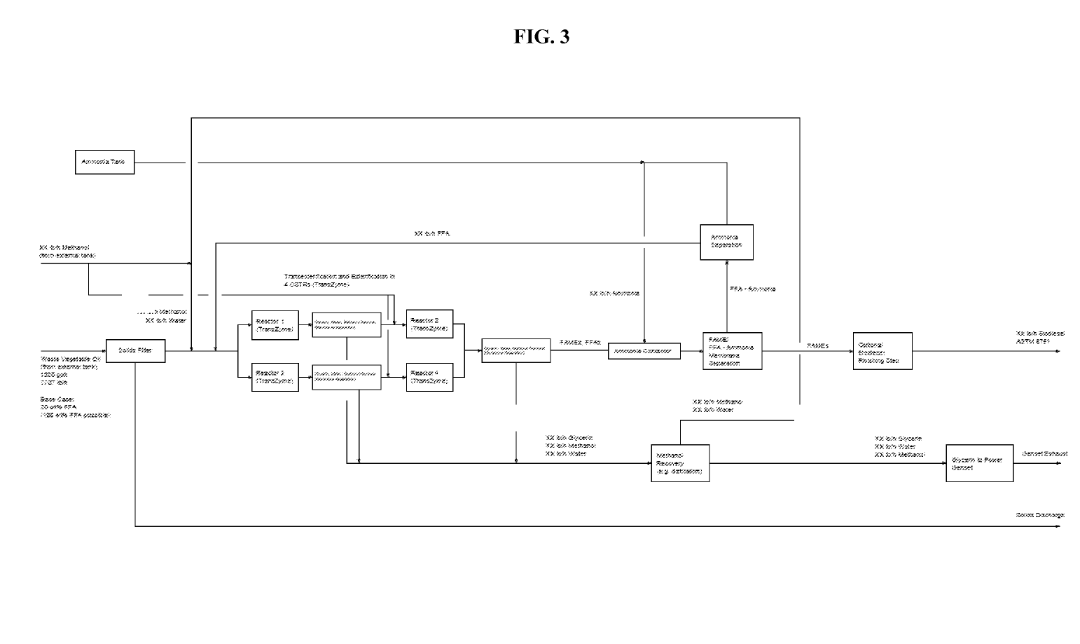

FIG. 3 depicts a specific process embodiment designed to convert, for example, 1000 gallons per hour of waste vegetable oil into biodiesel fuel. The process of FIG. 3 is capable of converting a wide range of feed rates of waste oil, and may be operated continuously, batch, or combination or variation thereof. The process steps for this embodiment are as follows.

Waste vegetable oil is heated via a plate and frame heat exchanger (for flowability) and filtered through a 1-micron filter bag in order to remove any foreign material.

Waste vegetable oil is then intensively mixed with methanol at stoichiometric levels (including a recycled free fatty acid stream and/or a recycled methanol stream) and then introduced to the first enzymatic-transesterification CSTR reactors (Reactors 1 and 3, in parallel).

After a specific retention time, such as about 2 hours, the flow from the CSTR Reactors 1 and 3 is directed to a membrane separator where glycerin, water, and methanol are separated from the FAME/FFA/TAG stream.

The glycerin, water, and methanol stream is further directed to another membrane separator where the glycerin/water and methanol are separated. The glycerin/water is directed to a genset (after an additional vacuum flash step to remove additional methanol which is recycled) where it is combusted to produce power for the operation. The methanol is recycled to be used at the start of the process.

The FAME/FFA/TAG stream is then mixed with additional methanol and directed to the second enzymatic-transesterification CSTR reactors (Reactors 2 and 4, in parallel). After a specific retention time, such as about 2 hours, the flow is directed to a second membrane separator and once again the glycerin, water, and methanol are separated from the now finished FAME/FFA stream. The glycerin/water is directed to the genset (after the additional vacuum flash step) and the residual methanol is recycled.

The FAME/FFA stream is now directed to an ammonia membrane contactor and then further directed to a membrane separator where the FAME is separated from the FFA/ammonia. The FAME is then directed to finished storage. In some cases, the FAME stream may be directed through a polishing step consisting of, but not limited to, an ion-exchange resin bed.

The FFA/ammonia stream is directed to a second membrane contactor where the ammonia is removed and sent back to storage to be used again.

The FFA stream is then directed back to the beginning of the process where it is intensively mixed with the incoming heated/filtered waste oil and methanol (prior to the first CSTR reactors).

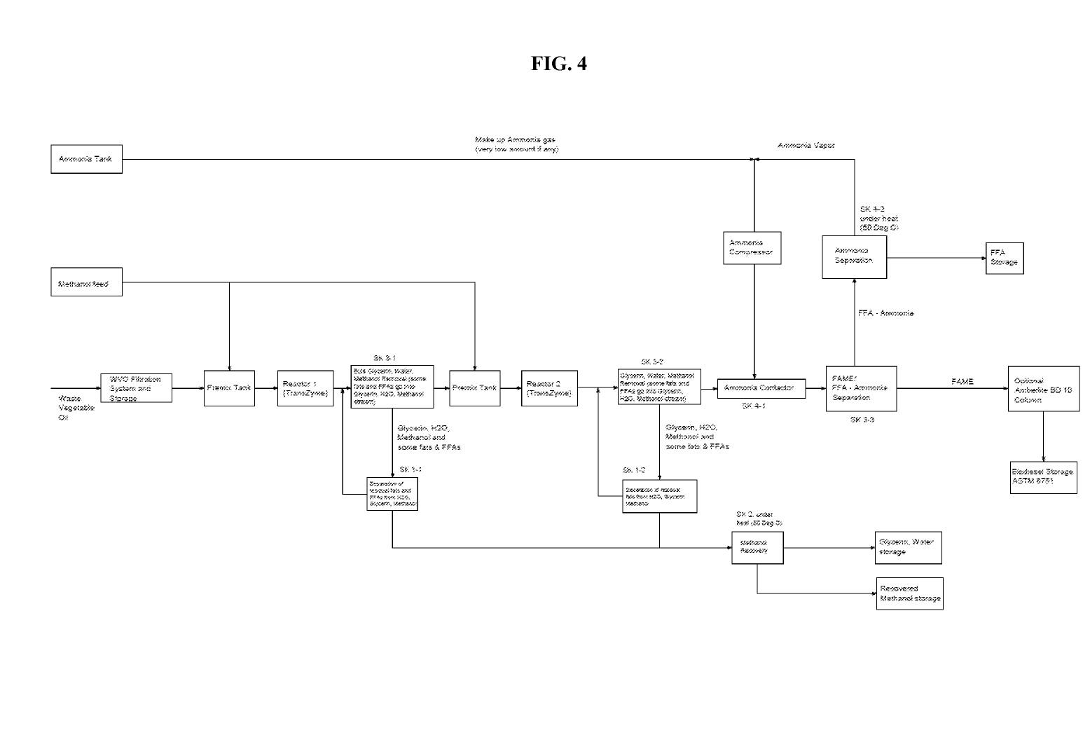

FIG. 4 depicts some process embodiments designed to convert waste vegetable oil into biodiesel fuel. The process of FIG. 4 may be employed at a wide variety of scales, including for example a laboratory-scale (e.g., about 1 gallon/hour of waste oil) in a batch, semi-continuous, or pseudo-continuous process. The process of FIG. 4 may also be employed at pilot, demonstration, or commercial scales such as 1000 gal/hr or more of waste oil continuously or semi-continuously fed. The process steps for the embodiments relating to FIG. 4 are generally similar to, and consistent with, the above descriptions with reference to FIGS. 1-3. The particular process (and system) of FIG. 4 does incorporate additional membrane separators, some in looped configurations (e.g., SK 3-1 with SK 1-1; refer to section “Membrane Separators” below for further membrane details). Also, note that rather than (or in addition to) being stored, the free fatty acids may be recycled to the front end of the process, similar to FIG. 3. Also, the glycerin may be sent to a genset rather than (or in addition to) being stored.

In some embodiments, the process is controlled or adjusted to attain certain biodiesel fuel properties. As is known, relevant biodiesel fuel properties can include flash point, cetane number, energy content, cloud point, gel point, pour point, glycerol content, water content, sediment content, ash content, sulfur content, nitrogen content, phosphorus content, pH, density, viscosity, lubricity, and so on.

In some embodiments, the biodiesel composition meets the specification set forth in ASTM D975 and/or ASTM D396-08c. In some embodiments, the composition further comprises a diesel fuel in a suitable blend, wherein the blend meets the specification set forth in ASTM D7467-08. In certain embodiments, the biodiesel composition produced according to the methods described herein conforms to EN 14214, which is a European standard that describes the requirements and test methods for fatty acid methyl esters utilized in biodiesel fuels. EN 14214 directly applies only to biodiesel fuel produced using methanol as the alcohol reactant.

In some variations of this invention, the process (and system for carrying out the process) is disposed substantially on a mobile processing unit. A mobile processing unit may be constructed on a vehicle (such as a truck) or portable means that may be moved (such as a trailer or skid). The combined heat and power from the genset may be utilized by the mobile processing unit, if desired, such as for on-board electronics.

While the present invention does not require that the process be mobile, several benefits arise from mobility. Variations of this invention that employ a mobile processing unit may be useful for business methods described and claimed in co-pending U.S. Patent App. No. 61/563,922 by Wheeler et al., filed Nov. 28, 2011, for “METHODS AND SYSTEMS FOR CONVERTING FOOD WASTE OIL INTO BIODIESEL FUEL” which is commonly assigned with the instant patent application. U.S. Patent App. No. 61/563,922 is incorporated by reference herein in its entirety for all purposes.

For example, a mobile processing unit may enable a method of providing a service to cooking oil users, distributers, or other market participants, the method comprising:

(a) identifying a network comprising a plurality of customer distribution centers, wherein each of the customer distribution centers is associated with a plurality of users of cooking oil;

(b) at a first customer distribution center, collecting used cooking oil transported from at least some of the users associated with the first customer distribution center, and introducing the used cooking oil to a first storage unit;

(c) providing a mobile processing unit configured to convert used cooking oil into biodiesel fuel as set forth herein;

(d) transporting the mobile processing unit to the first customer distribution center;

(e) converting at least some of the used cooking oil contained in the first storage unit biodiesel fuel in the mobile processing unit while it is located on-site at the first customer distribution center; and

(f) optionally introducing at least some of the biodiesel fuel, directly or via a fuel blend, into one or more fleet vehicles associated with the first customer distribution center.

The biodiesel fuel produced may be introduced, directly or via a fuel blend, into one or more fleet vehicles located at, or otherwise associated with, a customer site. A customer site may consist of a refiner (manufacturer) of cooking oil, a supplier of cooking oil, a user of cooking oil, or a distribution center associated with a plurality of suppliers and/or users of cooking oil, in certain embodiments of this invention.

The entire process need not necessarily be disposed on the mobile processing unit. For example, in some embodiments, certain ancillary units or tanks such as a methanol tank, ammonia tank, polishing unit, or genset may be provided or available external to the mobile processing unit. Some equipment may be available both on the mobile processing unit as well as external to it, at a certain site. Preferably, when it is desired to provide a mobile processing unit, substantially all units and equipment except for tanks for the main feedstocks (oil and alcohol) are disposed on a common mobile unit. Various embodiments will be appreciated by a skilled artisan, regarding which components of the system may be disposed on the mobile processing unit or provided externally.

In preferred embodiments of the invention, substantially no wastewater is directly discharged in connection with production of the biodiesel fuel. The mobile processing unit may be operated to be self-sufficient for energy demand, and in some embodiments the mobile reactor does not require external utilities, or has a very low utility demand.

A wide range of process capacities is possible. In some embodiments, the process is capable of converting between 1 and 10,000 gallons per hour of a feed oil stream (gph) to biodiesel fuel, including about 10, 100, 250, 500, 750, 1,000, or 5,000 gph. Less than 1 gph, such as at laboratory scale, and higher than 10,000 gph at commercial scale, are also possible. When the process is disposed on a mobile processing unit, there will be engineering and practical limitations of the scale for a single unit. However, a plurality of processing units may be provided in a network so that a desired distributed capacity is realized.

The conventional wisdom is that very large scale is necessary for biorefinery process economics. In the context of the present invention, it has been discovered that by scaling down the individual processing units and directing them to where feedstock is located—for example, waste oil at customer sites or distribution centers—smaller scales (such as about 1,000 gph) can actually be preferable. This is particularly true in embodiments that employ membrane separators. That is, membrane units are known to scale linearly, as opposed to typical process apparatus that scale with an exponent less than 1. Scaling up membrane technology is usually done by replicating modules. Normally, this is a cost disadvantage. The present inventors, however, have realized that this situation can become an advantage by distributing processing units in an overall business system (cf. commonly owned U.S. Patent App. No. 61/563,922) where the processing units are mobile, feedstock-flexible, and energy-efficient.

### Enzyme Catalysts

Additional disclosure regarding enzymes, as applicable to preferred embodiments of the invention, will now be provided. Generally speaking, suitable enzymes are commercially available although the selection of particular enzymes is not regarded as obvious.

In some embodiments, the esterification-transesterification catalyst is a free or immobilized enzymatic catalyst or mixture of enzymes. Contrary to alkaline catalysts, enzymes do not form soaps and can esterify both free fatty acids and triglycerides in one step without the need of a subsequent washing step. Enzymes are potentially useful compared to alkaline or acid catalysts, because they are more compatible with variations in the quality of the raw material and are reusable; can produce biodiesel fuel in fewer process steps using less energy and with drastically reduced amount of wastewater; and can yield a higher quality of glycerin.

Interfacial enzymes are a class of enzymes that comprise two domains in their proteinous structure; the first is a hydrophilic domain, while the second is a hydrophobic domain. This unique feature imparts this class of enzymes to favor the interfacial area once it is present in a two-phase system. Under these conditions, the active conformation is formed where the hydrophilic domain of the enzyme molecules faces the aqueous layer while the hydrophobic domain faces the hydrophobic layer.

Preferably, the esterification and transesterification enzymes possess enzymatic activity, under the applicable reaction conditions, selected from the group consisting of lipase activity, phospholipase activity, and esterase activity.

Lipases and phospholipases are known interfacial enzymes that express their catalytic activity once present in an interfacial system. Lipases (triacylglycerol hydrolase E.C. 3.1.1.3) are defined as hydrolytic enzymes that act on the ester linkage in triacylglycerol in aqueous systems to yield free fatty acids, partial glycerides, and glycerin. Phospholipases also belong to the class of hydrolytic enzymes; however, they cleave specifically the ester linkage of phospholipids present in aqueous systems, to yield free fatty acids, lysophospholipids, glycerophospholipids, phosphatidic acid, and free alcohol, depending on the type of phospholipase. Esterases are hydrolase enzymes that split esters into an acid and an alcohol in a hydrolysis chemical reaction.

Lipases from bacteria and fungi are the most commonly used for esterification or transesterification. In some embodiments, lipase, phospholipase, or esterase enzymes are derived from Candida antarctica, Candida rugosa, Rhizomucor miehei, Pseudomonas, Rhizopus niveus, Mucor javanicus, Rhizopus oryzae, Aspergillus niger, Penicillium camernbertii, Alcaligenes, Burhholderia, Thermomyces lanuginosa, Chromobacterium viscosum, papaya seeds, and pancreatin.

Immobilization of enzymes can be accomplished by a vast number of techniques basically aiming at reducing the cost contribution of enzymes in the overall process, facilitating the recovery of enzymes (or avoiding the loss of enzymes from the reactor in the first place), and enabling continuous operation of the process.

Immobilization techniques include physical adsorption of enzymes to solid supports, such as silica and insoluble polymers; adsorption on ion-exchange resins; covalent binding of enzymes to a solid support material, such as epoxidated inorganic or polymer supports; entrapment of enzymes in a growing polymer; confinement of enzymes in a membrane reactor or in semi-permeable gels; and/or cross-linking enzyme crystals or aggregates.

Typically, a major drawback of lipases and phospholipases is their low tolerance towards hydrophilic substrates, particularly short-chain alcohols (such as methanol) and short-chain fatty acids (below C₄). It is believed that short-chain alcohols and short-chain fatty acids, such as methanol and acetic acid, are responsible for detaching essential water molecules from the quaternary structure of those enzymes, leading to their denaturation and consequently loss of their catalytic activity.

Therefore, enzymes that have at least some tolerance to C₁-C₆ alcohols, and especially methanol, are desirable. One such enzyme is TransZyme, which is commercially available from Transbiodiesel (Shfar-Am, Israel).

In some embodiments, enzymes are modified interfacial enzymes immobilized on a solid support, wherein the enzymes are surrounded by a hydrophobic microenvironment. The enzymes are thereby protected from deactivation and/or aggregation in the presence of hydrophilic agents, substrates, and/or reaction products. The modified interfacial enzymes may be protected by being coated with covalently bonded lipid groups.

The support may be capable of binding the enzyme by adsorption or by covalent binding to functional groups. The support may be organic or inorganic, and is preferably selected from the group consisting of inorganic supports such as silica and alumina, and organic supports such as polymer-based supports. The support may contain active functional groups such as epoxy or aldehyde groups and ionic groups. The support may be an ion exchange resin. The lipid epoxide may be selected from fatty acids, fatty acid alkyl esters, sugar fatty acid esters, medium- and long-chain alkyl glucosides, phospholipids, polyethylene glycol derivatives, and quaternary ammonium salts. The modified interfacial enzyme may be protected by being immobilized on or embedded in a hydrophobic solid support which supplies the hydrophobic micro-environment.

Enzyme supports may be selected, for example, from Amberlite series (Rohm & Haas, US); Duolite series (Rohm & Haas); Amberlyst A-21 (Rohm & Haas); Dowex monosphere 77 (Dow Chemical, US); Dowex optipore L493 (Dow Chemical); Dow styrene DVB (Dow Chemical); MTO Dowex optipore SD-2 (Dow Chemical); Dowex MAC-3 (Dow Chemical); Purolite A109 (Purolite, US); and Sepabeads (Mitsubishi Chemical, Japan).

### Membrane Separators

Preferred embodiments of the invention employ membrane separators in several places within the process (e.g., FIG. 3). Any known membrane configuration (geometry) may be employed, including hollow fibers, flat sheets, or spiral-wound membranes. Membranes are commercially available although the selection of particular membranes is not regarded as obvious.

In some embodiments, membrane separators are provided by Separation Kinetics, Inc. (Eden Prairie, Minn., US). Separation Kinetics utilizes plasma polymerization processing and other coating techniques in the development of special membranes for gas and liquid separation applications. Plasma polymerization is a chemical bonding and surface modification technology used to develop membranes to achieve specific functionalities. Surfaces can be made wetable, non-fouling, slippery, highly cross-linked, reactive, reactable, or catalytic. Polymeric coatings impart added strength and durability. Surfaces can be hydrophobic or hydrophilic, made to provide oxidative resistance, and made to allow for customized membrane transport characteristics. (Ref.: http://separationkineticsinc.com, which is incorporated by reference as of the filing date of the present patent application.)

Reference is made to the exemplary process shown in FIG. 4, and the labels depicted therein. Membrane separation units SK 3-1 and 3-2 are designed and operated to separate glycerin/water/methanol from FAME/TAG/FFA. Membrane separation units SK 1-1 and 1-2 are designed and operated to separate residual FAME/TAG/FFA from glycerin/water/methanol, where the residual FAME/TAG/FFA is looped back to the feed to unit SK 3-1 (from SK 1-1) or SK 3-2 (from SK 1-2).

Membrane unit SK 4-1 is a membrane contactor designed and operated to introduce ammonia into the FAME/FFA stream from unit SK 3-2. Membrane unit SK 4-2 is a membrane contactor designed to remove ammonia from the FFA/ammonia stream derived from unit SK 3-3. Membrane unit SK 4-2 may be operated under heat, such as at a temperature of about 50° C. Membrane separation unit SK 3-3 is designed and operated to remove FAME from the FAME/FFA/ammonia stream from unit SK 4-1.

Membrane separation unit SK 2 is designed and operated to remove methanol from the glycerin/water/methanol stream from units SK 1-1 and 1-2 (or directly from units SK 3-1 and 3-2, if the loops are not used). Membrane unit SK 2 may be operated under heat, such as at a temperature of about 50° C.

In certain embodiments, a membrane separator may comprise a microporous polyethylene or polypropylene substrate with an unwoven backing. The substrate is coated with an extremely thin but chemically bonded and physically sturdy layer, which is formed by gas-phase deposition. For example, a plasma polymer of methane may be deposited (“methane coating”).

With reference to FIG. 4, the SK 1-1 and 1-2 membranes may be coated with an acrylic acid/methane coating. The SK 2 membrane may be coated with hexafluoropropylene. The SK 3-1 and 3-2 membranes may be coated with methane coatings. The SK 4-1 and SK 4-2 membranes may be coated with a disiloxane/air coating. These are indicative of the types of coatings that may be employed in various embodiments.

The ammonia loop involves membrane units SK 4-1, 4-2, and 3-3. Without being limited to any particular hypothesis, it is believed that ammonia actually creates a complex with free fatty acid by attaching at the molecular level. It is thought that the fatty acid and the base (ammonia in this example) chemically or physically bond, absorb, adsorb, or otherwise interact with each other. The fatty acid-base complex may then be conveniently separated from fatty acid alkyl esters in unit SK 3-3. Then, through heating or other means, the base may be recovered in unit SK 4-2 from the fatty acid-base complex to generate free fatty acids for optional recycling, or storage as indicated in FIG. 4. Unit SK 4-2 should be coupled to a gas contactor for ammonia removal. An exemplary gas contactor known in the art that may be adapted for this use is described in U.S. Pat. No. 5,439,736, which is incorporated by reference herein.

Ammonia and any of its derivatives, such as ammonium hydroxide which may form from reaction with water present, is a preferred base. Although other bases would be effective for this separation scheme, it is noted that ammonia is particularly convenient because it is normally a gas and can be released upon heating (e.g., to 50° C.) from membrane unit SK 4-2. If there are no free fatty acids present in the FAME/FFA stream, the ammonia goes right back into the ammonia loop. Only the ammonia needed for a particular FFA level in the stream is used, which is efficient from a processing standpoint.

A variation of the present invention is premised on the recognition that the overall process may become more efficient if reactors and separators could somehow be combined. Such integration may be achieved with membrane reactors.

In some embodiments, enzymes are immobilized on the same membranes (e.g., units SK 3-1 and 3-2 in FIG. 4) that are employed for some desired separations. The desired chemistry is carried out by immobilized enzymes, while at the same time, the desired separation is achieved by the membranes which serve as a support for the enzymes. A significant advantage of this scheme is that by continuously separating products from reactants, the equilibrium conversion is no longer a constraint. It is thus possible, if not preferable, to employ a single reactor/separator rather than a series of reactors and separators.

In some embodiments of this variation, a process for producing biodiesel fuel comprises the steps of:

(a) providing a feed stream comprising glycerides and free fatty acids;

(b) optionally filtering out at least a portion of solids contained in the feed stream;

(c) introducing the feed stream and an alcohol (such as methanol) into a membrane reactor configured with immobilized esterification and transesterification enzymes supported on membranes, wherein the membrane reactor is operated at effective reaction conditions to react at least a portion of the free fatty acids with the alcohol to produce fatty acid alkyl esters and water, and to react at least a portion of the glycerides with the alcohol to produce fatty acid alkyl esters and glycerin, thereby producing an in situ mixture comprising fatty acid alkyl esters, glycerin, water, and alcohol; and wherein the membrane reactor, under effective reaction conditions, continuously separates at least some glycerin, water, and alcohol, thereby producing an intermediate stream comprising fatty acid alkyl esters and unreacted free fatty acids;

(d) optionally further reacting, separating, or treating the intermediate stream; and

(e) introducing the intermediate stream and a base (such as ammonia) to a fatty acid alkyl ester recovery unit operated to contact the base with the unreacted free fatty acids to produce a fatty acid-base complex, and separate the fatty acid-base complex from the fatty acid alkyl esters, thereby generating a crude product stream comprising the fatty acid alkyl esters.

Other variations of this invention relate to diesel fuel compositions. A diesel fuel may be produced, including this biodiesel fuel compositions provided herein, and used in any known diesel fuel application. Preferred compositions are capable of burning in an internal combustion engine. In some of these embodiments, the blended diesel fuel meets the specification set forth in ASTM D7467-08.

All publications, patents, and patent applications cited in this specification are incorporated herein by reference in their entirety as if each publication, patent, or patent application was specifically and individually put forth herein. All ASTM or EN specifications recited herein are also incorporated by reference. Additionally, all relevant U.S. laws, rules, procedures, tax code, and the like, with respect to biodiesel fuel production, distribution, and use, are incorporated by reference herein.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent that there are variations of the invention, which are within the spirit of the disclosure or equivalent to the inventions found in the appended claims, it is the intent that this patent will cover those variations as well. The present invention shall only be limited by what is claimed.

1. A process for producing biodiesel fuel, said process comprising:
(a) providing a feed stream comprising glycerides and free fatty acids;
(b) optionally filtering out at least a portion of solids contained in said feed stream;
(c) introducing said feed stream and an alcohol into a first reactor, operated at effective first reaction conditions and including a first effective esterification and transesterification catalyst, or mixture of such catalysts, to react at least a portion of said free fatty acids with said alcohol to produce fatty acid alkyl esters and water, and to react at least a portion of said glycerides with said alcohol to produce fatty acid alkyl esters and glycerin, thereby producing a first intermediate stream comprising fatty acid alkyl esters, glycerin, water, and alcohol;
(d) introducing at least a portion of said first intermediate stream to a first separator operated to remove at least some glycerin, water, and alcohol, thereby producing a second intermediate stream comprising fatty acid alkyl esters and unreacted free fatty acids;
(e) optionally further reacting, separating, or treating said second intermediate stream; and
(f) introducing said second intermediate stream and a base to a fatty acid alkyl ester recovery unit operated to contact said base with said unreacted free fatty acids to produce a fatty acid-base complex, and separate said fatty acid-base complex from said fatty acid alkyl esters, thereby generating a crude product stream comprising said fatty acid alkyl esters.

2. The process of claim 1, wherein said glycerides consist essentially of triglycerides.

3. The process of claim 1, wherein said feed stream includes an oil derived from a food source selected from the group consisting of soybeans, corn, canola, rice, olive, coconut, cottonseed, palm, peanut, rapeseed, safflower, sesame, sunflower, pumpkin, grape, animal fat, and combinations thereof.

4. The process of claim 1, wherein said feed stream includes a waste oil.

5. The process of claim 1, wherein said feed stream includes an inedible oil.

6. The process of claim 1, wherein said feed stream includes an oil derived from an energy crop.

7. The process of claim 1, wherein said alcohol is selected from the group of C₁-C₆ alcohols, and combinations thereof.

8. (canceled)

9. The process of claim 1, said process further comprising intensively mixing said alcohol with said feed stream and with recycled free fatty acids, if any.

10-12. (canceled)

13. The process of claim 1, wherein said first effective esterification and transesterification catalyst is a single catalyst with both esterification and transesterification activity.

14. The process of claim 1, wherein said first effective esterification and transesterification catalyst is a mixture of catalysts collectively including esterification and transesterification activity.

15. The process of claim 1, wherein said first effective esterification and transesterification catalyst is an alkali catalyst.

16. The process of claim 1, wherein said first effective esterification and transesterification catalyst is an acid catalyst.

17. The process of claim 1, wherein said first effective esterification and transesterification catalyst is an enzymatic catalyst with activity, at said effective first reaction conditions, selected from the group consisting of lipase activity, phospholipase activity, esterase activity, and combinations thereof.

18. The process of claim 17, wherein said enzymatic catalyst is immobilized.

19. The process of claim 17, wherein said enzymatic catalyst is tolerant to said alcohol.

20. (canceled)

21. (canceled)

22. The process of claim 1, wherein said first separator is selected from the group consisting of a distillation unit, a flash evaporation unit, a centrifuge, a filter, membranes, and any combinations thereof.

23. The process of claim 22, wherein said first separator comprises membranes.

24. The process of claim 1, wherein said fatty acid alkyl ester recovery unit is selected from the group consisting of a distillation unit, a flash evaporation unit, a centrifuge, a filter, membranes, and any combinations thereof.

25. The process of claim 24, wherein said fatty acid alkyl ester recovery unit comprises membranes.

26. The process of claim 1, wherein said base introduced in step (f) consists essentially of ammonia and derivatives thereof.

27. The process of claim 1, said process further comprising recovering said base from said fatty acid-base complex to generate free fatty acids.

28. The process of claim 27, wherein said fatty acid alkyl ester recovery unit includes a first membrane contactor, a membrane separator for separating said fatty acid alkyl esters from said fatty acid-base complex, and a second membrane contactor for recovering said base.

29. The process of claim 1, said process further comprising introducing glycerin, water, and alcohol from said first separator to an alcohol recovery unit operated to remove alcohol, thereby producing a glycerin stream comprising glycerin and water.

30. The process of claim 29, wherein said alcohol recovery unit is selected from the group consisting of a distillation unit, a flash evaporation unit, a centrifuge, membranes, molecular sieves, and any combinations thereof.

31. The process of claim 30, said process further comprising recycling at least a portion of alcohol removed from said alcohol recovery unit back to step (c).

32. The process of claim 29, said process further comprising combusting at least some of said glycerin stream in a genset to produce electrical power and thermal heat.

33. The process of claim 1, said process further comprising introducing glycerin and alcohol from said first separator to a genset to produce electrical power and thermal heat.

34. The process of claim 1, said process further comprising introducing glycerin, alcohol, and water from said first separator to a fuel cell genset to produce electrical power and thermal heat.

35. The process of claim 1, said process further comprising conveying said crude product stream to storage.

36. The process of claim 1, said process further comprising subjecting said crude product stream to one or more polishing steps to produce a product stream comprising said fatty acid alkyl esters.

37. The process of claim 36, wherein said polishing steps include ion exchange.

38. A process for producing biodiesel fuel, said process comprising:
(a) providing a feed stream comprising glycerides and free fatty acids;
(b) optionally filtering out at least a portion of solids contained in said feed stream;
(c) introducing said feed stream and an alcohol into a first reactor, operated at effective first reaction conditions and including a first effective esterification and transesterification catalyst, or mixture of such catalysts, to react at least a portion of said free fatty acids with said alcohol to produce fatty acid alkyl esters and water, and to react at least a portion of said glycerides with said alcohol to produce fatty acid alkyl esters and glycerin, thereby producing a first intermediate stream comprising fatty acid alkyl esters, glycerin, water, and alcohol;
(d) introducing at least a portion of said first intermediate stream to a first separator operated to remove at least some glycerin, water, and alcohol, thereby producing a second intermediate stream comprising fatty acid alkyl esters and unreacted free fatty acids;
(e) introducing said second intermediate stream and an alcohol to a second reactor, operated at effective second reaction conditions and including a second effective esterification and transesterification catalyst, or mixture of such catalysts, to react free fatty acids contained therein with said alcohol to produce fatty acid alkyl esters and water, and/or to react glycerides contained therein with said alcohol to produce fatty acid alkyl esters and glycerin, thereby producing a third intermediate stream comprising fatty acid alkyl esters, glycerin, water, and alcohol;
(f) introducing at least a portion of said third intermediate stream to a second separator operated to remove at least some glycerin, water, and alcohol, thereby producing a fourth intermediate stream comprising fatty acid alkyl esters and unreacted free fatty acids; and
(g) introducing said fourth intermediate stream and a base to a fatty acid alkyl ester recovery unit operated to contact said base with said unreacted free fatty acids to produce a fatty acid-base complex, and separate said fatty acid-base complex from said fatty acid alkyl esters, thereby generating a crude product stream comprising said fatty acid alkyl esters.

39-75. (canceled)

76. A process for producing biodiesel fuel, said process comprising:
(a) providing a feed stream comprising glycerides and free fatty acids;
(b) optionally filtering out at least a portion of solids contained in said feed stream;
(c) introducing said feed stream and an alcohol into a membrane reactor configured with immobilized esterification and transesterification enzymes supported on membranes, wherein said membrane reactor is operated at effective reaction conditions to react at least a portion of said free fatty acids with said alcohol to produce fatty acid alkyl esters and water, and to react at least a portion of said glycerides with said alcohol to produce fatty acid alkyl esters and glycerin, thereby producing an in situ mixture comprising fatty acid alkyl esters, glycerin, water, and alcohol; and wherein said membrane reactor, under effective reaction conditions, continuously separates at least some glycerin, water, and alcohol, thereby producing an intermediate stream comprising fatty acid alkyl esters and unreacted free fatty acids;
(d) optionally further reacting, separating, or treating said intermediate stream; and
(e) introducing said intermediate stream and a base to a fatty acid alkyl ester recovery unit operated to contact said base with said unreacted free fatty acids to produce a fatty acid-base complex, and separate said fatty acid-base complex from said fatty acid alkyl esters, thereby generating a crude product stream comprising said fatty acid alkyl esters.

77-90. (canceled)

91. The process of claim 1, wherein said process is disposed substantially on a mobile processing unit.

92. The process of claim 1, wherein said process is continuous or semi-continuous.

93. The process of claim 1, wherein said process is a batch process.

94. The process of claim 1, wherein said process converts between 100 and 10,000 gallons per hour of said feed stream.

95. The process of claim 94, wherein said process converts at least 1,000 gallons per hour of said feed stream.

96. The process of claim 1, wherein substantially no wastewater is directly discharged in connection with production of said biodiesel fuel.

97. The process of claim 1, wherein said process is energy self-sufficient and requires substantially no external utilities.

98. (canceled)

99. (canceled)
